(19) United States
(12) Patent Application Publication
(10) Pub. No.: US 2026/0078074 A1
(43) Pub. Date: Mar. 19, 2026

(54) INTEGRATED GREEN METHANOL SYNTHESIS APPARATUS WITH HEAT TRANSFER, HEAT STORAGE AND HEAT EXCHANGE BY MOLTEN SALT

(71) Applicant: BEIJING MINLI ENERGY STORAGE TECHNOLOGY CO., LTD., BEIJING (CN)

(72) Inventors: SHUGUANG ZHAO, BEIJING (CN); SHIHUI MU, BEIJING (CN); JIANXIN WANG, BEIJING (CN); XINGYE CHEN, BEIJING (CN)

(21) Appl. No.: 19/325,976

(22) Filed: Sep. 11, 2025

(30) Foreign Application Priority Data
Sep. 14, 2024 (CN) 202411287770.9

(51) Int. Cl. C07C 29/152; C07C 31/04

(52) U.S. Cl. CPC C07C 29/152; C07C 31/04

(57) ABSTRACT

An integrated green methanol synthesis apparatus with heat transfer, heat storage and heat exchange by molten salt is provided. A molten salt with a wide temperature range, a high specific heat capacity and an atmospheric pressure is used as a heat transfer medium, a heat storage medium circulation system is provided to continuously transfer reaction heat generated in an inner cavity to an outer cavity to heat a feed gas, and the heat generated in the inner cavity is stored in the molten salt medium in the form of sensible heat, so that the green methanol synthesis apparatus achieves integration of functions of heat transfer, heat storage and heat exchange and can effectively solve the problems in the existing methanol synthesis technology, such as low temperature of the feed gas entering the synthesis apparatus, a large number of accessory devices, and large space occupation.

### CROSS-REFERENCE TO RELATED APPLICATION

This patent application claims the benefit and priority of Chinese Patent Application No. 202411287770.9 filed with the China National Intellectual Property Administration on Sep. 14, 2024, the disclosure of which is incorporated by reference herein in its entirety as part of the present application.

### TECHNICAL FIELD

The present disclosure belongs to the field of green methanol synthesis technology, and particularly relates to an integrated green methanol synthesis apparatus with heat transfer, heat storage and heat exchange by molten salt.

### BACKGROUND

Methanol, as the simplest saturated monohydric alcohol, is an important chemical raw material and clean fuel, which can be used in industries such as organic chemical raw materials, new energy, and medicines. Methanol can be obtained in three ways, including hydrolysis of chloromethane, oxidation of methane and a feed gas method. The first two methods have not been widely implemented in industry due to limitations in methanol production. At present, the industry often uses the feed gas method to produce methanol. The main components of a feed gas include carbon monoxide, hydrogen and carbon dioxide. Due to the development and application of methanol fuels, the demand for methanol at home and abroad is increasing day by day. The method of producing methanol from the feed gas provides a new way for carbon dioxide capture and consumption, and actively responds to the “double carbon” policy in China. Therefore, the technology of producing methanol by the feed gas method has gradually begun to be valued.

The technology of synthesizing methanol from the feed gas has the following deficiencies. (1) In the conventional art, the feed gas needs to be heated by a separate heater or preheated by a heat exchanger before entering the synthesis apparatus. The temperature of the preheated feed gas entering the synthesis apparatus is lower than the temperature of a catalyst bed inside the synthesis apparatus. The low temperature of the feed gas leads to an increase in side reactions inside the synthesis apparatus. The method of heating the feed gas has certain requirements for the covering area and needs high investment costs. (2) Under the specific production conditions of green methanol, a fixed tube-sheet heat exchange tube bundle built in the synthesis apparatus has a high-temperature heat alternating load of reaction exotherm, which is prone to the possibility of heat exchange tubes being detached from fixed tube-sheets to damage the heat exchange tube bundle. If an accident of damage to the heat exchange tube bundle occurs, it will result in soft water inside the heat exchange tubes leaking into the synthesis apparatus, causing major safety accidents such as failure of the catalyst and overpressure caused by instant vaporization of water. (3) Under restriction of the specific reaction temperature conditions of green methanol, steam pressure corresponding to the temperature of saturated steam in a steam drum is lower, resulting in lower output steam grade, limitations on the recovered heat during secondary utilization, failing to produce superheated steam, and the like. The situation of high energy consumption and low utilization is seriously inconsistent with energy utilization rules. (4) Since the process of synthesizing methanol from the feed gas is an exothermic process, the existing methanol synthesis apparatus adopts the form of high-pressure water cooling, in which the internal reaction temperature of the synthesis apparatus is controlled by controlling the pressure of the external steam drum. Such a temperature control method requires the installation of a steam drum and a corresponding chemical dosing apparatus, which has certain limitations for users with smaller sites and needs high equipment investment costs. (5) The existing synthesis apparatus achieves heat transfer by water cooling, which needs to replenish a large amount of water resources for heat exchange. Because the use of tap water will corrode internal components of the synthesis apparatus, softened water or pure water is needed and a separate water production apparatus needs to be provided, resulting in an increase in water production costs and operating expenses. (6) The heat released by the synthesis of methanol from the same molar of carbon monoxide and carbon dioxide differs by nearly twice, and the former releases much more heat than the latter. When the amount of carbon monoxide contained in the feed gas is high, the strong reaction heat generated rapidly inside the synthesis apparatus cannot be transferred, resulting in overheating inside the synthesis apparatus and deactivation of the catalyst, which is not conducive to the control of reaction operations. In order to avoid this phenomenon, the existing treatment method is to convert carbon monoxide into carbon dioxide and hydrogen by adding water vapor, and then remove the excess carbon dioxide. However, this treatment method reduces the content of synthesized methanol due to the reduction of carbon elements involved in reactions, generates emission of carbon dioxide, and requires separate conversion equipment.

In view of this, the present disclosure proposes an integrated green methanol synthesis apparatus with heat transfer, heat storage and heat exchange by molten salt.

### SUMMARY

In view of the deficiencies of the conventional art, the problem to be solved by the present disclosure is to provide an integrated green methanol synthesis apparatus with heat transfer, heat storage and heat exchange by molten salt. According to the synthesis apparatus, a molten salt with a wide temperature range, a high specific heat capacity and an atmospheric pressure is used as a heat transfer medium, a heat storage medium circulation system is provided to continuously transfer reaction heat generated in an inner cavity to an outer cavity to heat a feed gas, and the heat generated in the inner cavity is stored in a molten salt medium in the form of sensible heat, so that the green methanol synthesis apparatus achieves integration of functions of heat transfer, heat storage and heat exchange and can effectively solve the problems in the existing methanol synthesis technology, such as low temperature of the feed gas entering the synthesis apparatus, a large number of accessory devices, and large space occupation. The molten salt with a high specific heat capacity is used to sufficiently absorb the high heat generated inside the synthesis apparatus to maintain stable operation of the main reaction inside the synthesis apparatus, avoiding the circumstance of converting carbon monoxide to carbon dioxide and then removing the carbon dioxide in order to maintain an internal temperature of the synthesis apparatus in the conventional art, avoiding the issue of carbon dioxide emission and reducing the loss of feed gas, thereby increasing the yield of methanol.

In order to achieve the object described above, the present disclosure uses the following technical solutions.

An integrated green methanol synthesis apparatus with heat transfer, heat storage and heat exchange by molten salt includes a reaction shell, and a heat exchange assembly, a cavity and a feed gas distribution assembly disposed inside the reaction shell; where the reaction shell includes, from top to bottom, a floating head, a reaction barrel and a lower head; a feed gas reaction outlet and a catalyst feed inlet are arranged on the floating head; a catalyst discharge outlet and a heat storage medium outlet are arranged on the lower head; the reaction barrel is composed of an inner barrel and an outer barrel as a whole;
the cavity includes an inner cavity formed by an inner wall of the inner barrel and an inner wall of the floating head, and an outer cavity formed by an outer wall of the inner barrel, an inner wall of the outer barrel and an inner wall of the lower head;
the heat exchange assembly includes a heat storage medium tube bundle in the inner cavity and a feed gas tube bundle in the outer cavity;
the feed gas distribution assembly is located at a bottom of the inner cavity enclosed by the inner barrel;
a heat storage medium inlet is arranged on the inner barrel, and the heat storage medium inlet is located below a joint between the inner barrel and the floating head and above the outer barrel;
a feed gas inlet is arranged at an upper part of the outer barrel;
the feed gas tube bundle is arranged in a coiling form inside the outer cavity from top to bottom, with an upper end being a coil start end connected to the feed gas inlet, and a lower end being a coil tail end connected to the feed gas distribution assembly within the inner barrel; each circle of a coil of the feed gas tube bundle is fixed to the inner wall of the outer barrel and/or the outer wall of the inner barrel via some of support members;
the heat storage medium tube bundle is located in the inner cavity of the inner barrel and arranged in a coiling form from top to bottom along an intermediate axis of the reaction shell, with an upper end being a coil start end connected to the heat storage medium inlet, and a lower end being a coil tail end passing through the inner barrel to be in communication with the outer cavity; each circle of a coil of the heat storage medium tube bundle is fixed to an inner wall of the inner barrel via some of the support members; and
the heat storage medium outlet is connected to an inlet of a circulating pump via an external pipeline, and an outlet of the circulating pump is connected to the heat storage medium inlet via the external pipeline; and both the outer cavity and an interior of the heat storage medium tube bundle are filled with a molten salt heat storage medium.

In some embodiments, a flange is welded to a lower end of the floating head, a counter flange matching with the flange at the lower end of the floating head is welded to an upper end of the inner barrel, and the flange of the floating head and the counter flange of the inner barrel fixedly connect the floating head and the inner barrel by means of bolts, nuts and washers, which belongs to a detachable connection; the outer barrel is fixedly welded to and cover an outer layer of the inner barrel, and a distance from an upper edge of the outer barrel to an end face of the flange of the inner barrel is 30 cm-50 cm; a lower end of the outer barrel is rigidly connected to the lower head by welding; and the inner barrel is in a form of a cylinder with a lower end closed and the upper end open.

In some embodiments, the feed gas distribution assembly includes a feed gas bend tube, a feed gas straight tube, and a feed gas distribution head connected by welding, the feed gas bend tube is connected to the coil tail end at the lower end of the feed gas tube bundle; and preferably, the feed gas distribution head is of a spherical mesh structure.

In some embodiments, ones of the support members at a same height are uniformly distributed in a circumferential direction, the support members have horizontal support surfaces, and the coil of the feed gas tube bundle and the coil of the heat storage medium tube bundle are fixed onto the horizontal support surfaces by means of semicircular clamps; and each of the support members within the outer cavity has a length equal to a width of the outer cavity or extending from the outer wall of the inner barrel to an outer side of a corresponding tube of the coil of the feed gas tube bundle, each of the support members within the inner cavity has a length extending from the inner wall of the inner barrel to an inner side of a corresponding tube of the coil of the heat storage medium tube bundle, and the support members are capable of supporting the feed gas tube bundle/the heat storage medium tube bundle.

In some embodiments, a straight tube section of the catalyst discharge outlet passes through the lower head and a bottom end of the inner barrel in sequence and is in communication with the inner cavity, the straight tube section of the catalyst discharge outlet is connected with the lower head and the bottom end of the inner barrel by welding at contact positions; the catalyst discharge outlet is blocked by means of a blind plate during operation of the synthesis apparatus; and
a catalyst is filled in the inner cavity formed by the inner wall of the inner barrel and the inner wall of the floating head, with a packing factor up to 80%.

In some embodiments, diagonal braces are disposed within the outer cavity to provide a supporting force for the inner barrel, the diagonal braces are uniformly arranged around an outer surface of the inner barrel, and a specific number of the diagonal braces is set by checking a total weight of the inner barrel and a load inside the inner barrel; and
each of the diagonal braces includes a lower pad, an upper pad, and a diagonal stay, the lower pad is made as a pad having a curvature consistent with that of an inner surface of the lower head, the lower pad is connected to the lower head by full-length welding, the upper pad is made as a pad having a curvature consistent with that of the outer surface of the inner barrel, the upper pad is connected to the inner barrel by full-length welding, and two ends of the diagonal stay are fittingly welded to the upper pad and the lower pad respectively; and preferably, an included angle between the diagonal brace and the outer surface of the inner barrel is 30°-45°.

In some embodiments, a bottom of the inner barrel is constructed as a dome structure to facilitate discharging of a catalyst.

The molten salt heat storage medium is of a binary salt or a ternary salt. The molten salt selected needs to satisfy the requirements for properties such as wide temperature range, high specific heat capacity, good fluidity, low melting point and high boiling point.

In some embodiments, an interval between upper and lower adjacent tubes of each of the feed gas tube bundle and the heat storage medium tube bundle is of 3 cm-6 cm.

In some embodiments, a multi-point thermocouple is arranged from top to bottom along an inner surface of the inner barrel 6 and configured to measure an internal temperature of the inner barrel in real time; and the circulating pump 19 is a high-temperature-resistant variable-frequency molten salt pump, and a frequency of the circulating pump 19 is controlled by receiving temperature signals from the multi-point thermocouple to control a flow rate of the molten salt heat storage medium in the heat storage medium tube bundle 11 and adjust a heat transfer rate.

In some embodiments, the heat storage medium inlet is disposed on a side wall of the outer surface of the inner barrel, between the upper edge of the outer barrel and the end face of the flange of the inner barrel, and at an upper right end of the reaction shell; and the heat storage medium inlet is welded to a wall surface of the inner barrel.

In some embodiments, the feed gas inlet is disposed on a side wall of an outer surface of the outer barrel, at an upper right end of the outer barrel, and directly below the heat storage medium inlet; and the feed gas inlet is welded to a wall surface of the outer barrel.

In some embodiments, the heat storage medium outlet is located at the lower right side of an outer surface of the lower head, penetrates the lower head and is welded at a position of contact with the lower head; and the heat storage medium outlet is located directly below the feed gas inlet and the heat storage medium inlet.

In some embodiments, the catalyst feed inlet is located at an angle of 45° on the right side of the floating head, the catalyst feed inlet and the floating head are connected by welding at contact positions, and the catalyst feed inlet is blocked by a blind plate during operation of the synthesis apparatus.

In some embodiments, the catalyst discharge outlet and the feed gas reaction outlet are installed at the intermediate axis of the reaction shell.

The feed gas flows in the feed gas tube bundle.

In some embodiments, the heat storage medium tube bundle, the outer cavity, the circulating pump and the external pipeline together form the heat storage medium circulation system.

Compared with the conventional technology, the present disclosure achieves the following beneficial effects.

1. The green methanol synthesis apparatus of the present disclosure is an integrated device coupled with heat transfer, heat storage and heat exchange. The green methanol synthesis apparatus is compared with the existing synthesis apparatus as follows. The existing synthesis apparatus needs to preheat or heat the feed gas by means of an external preheater or heater; as the synthesis apparatus of the present disclosure is provided with a preheating space composed of the outer cavity and provided with the heat storage medium circulation system, the heat storage medium absorbs the reaction heat in the inner cavity to heat the feed gas in the outer cavity, the entire heating process is performed within an independent synthesis apparatus, and there is no need to provide the external preheater or heater. The green methanol synthesis apparatus of the present disclosure is simple in structure and small in space occupation, and accordingly the entire methanol synthesis system is more economical, with the costs significantly decreased by approximately 10%-20%.

2. The heat transfer structure of the green methanol synthesis apparatus is compared with that of the existing synthesis apparatus as follows. The existing synthesis apparatus uses a built-in fixed tube-sheet heat exchange tube bundle for heat transfer, the tube bundle is in the form of fixed straight tubes, which are filled with high-pressure water inside for heat transfer. In this heat transfer mode, the pressure on the tubes is high, the form of the fixed straight tubes makes the tubes have poor ability to relieve thermal stress, and is prone to tube cracking, causing leakage of high-pressure water inside the tube bundle, and leading to deactivation of the catalyst in the synthesis apparatus. The leaked high-pressure water instantly vaporized, causing overpressure in the synthesis apparatus and thus causing major safety accidents. The present disclosure adopts the heat storage medium tube bundle as the heat transfer and heat storage fitting, which uses a coiling form, and the heat storage medium in the tube bundle is at atmospheric pressure. Tubes of the tube bundle withstand small pressure and can effectively relieve thermal stress through rotation curvature, avoiding the problem that the fixed tube-sheet heat exchange tube bundle in the conventional art is prone to cracking, and enhancing the stability of the methanol synthesis apparatus.

3. The green methanol synthesis apparatus of the present disclosure adopts the heat storage medium tube bundle filled with the circulating molten salt to transfer heat inside the synthesis apparatus, and accordingly, under normal circumstances, no replenishment is needed. However, the existing synthesis apparatus adopts the form of heat transfer by high-pressure water, which needs replenishment of a large amount of water resources for transferring the reaction heat inside the synthesis apparatus, and is restrictive for water-scarce areas. Moreover, in order to avoid corrosion of equipment, the cooling water used needs to be softened water or purified water, which leads to an increase in the operating costs and equipment investment of the conventional art for methanol synthesis. As the present disclosure is provided with the heat storage medium circulation system, the heat storage medium is recycled in the synthesis apparatus without the need of replenishment. According to the present disclosure, the conventional water phase change high-pressure heat transfer medium is changed to a molten salt sensible-heat atmospheric-pressure heat transfer medium. Moreover, a vaporization temperature of the molten salt heat transfer medium is much higher than a maximum reaction temperature in the synthesis apparatus, and accordingly the vaporization phenomenon is omitted, which can effectively solve the problems of internal leakage and overpressure of water vaporization after the internal leakage in the conventional art. Since the present disclosure uses the molten salt with a high specific heat capacity as the heat storage medium, it is possible to eliminate the dependence of the synthesis apparatus on water resources, remove the water production equipment, and reduce the investment costs, operating costs and construction cycle.

4. The green methanol synthesis apparatus of the present disclosure uses the heat storage medium circulation system to achieve temperature control of the entire synthesis apparatus. It is compared with the existing methanol synthesis apparatus as follows. In the conventional art, a final heat exchange temperature of the cooling water is mainly controlled by controlling the pressure of the external steam drum, thereby controlling the reaction temperature inside the synthesis apparatus. In the present disclosure, the heat storage medium circulation system in the atmospheric-pressure form is provided, the heat of the inner cavity is transferred to the outer cavity by means of the heat storage medium continuously, and the heat of the heat storage medium in the outer cavity is used for heating up the feed gas. The heat storage medium circulation system not only plays a role in preheating the feed gas but also meets the requirements for the temperature control effect of the inner cavity. Compared to the conventional art, the temperature control method of the present disclosure adopts an atmospheric-pressure circulation form, which enhances the safety of the system. Since the heat generated by the apparatus itself is used for heating up the feed gas, on the one hand, the input of external energy is reduced and self-generation and self-dissipation of heat is enabled; and on the one hand, the equipment such as the external steam drum and the corresponding chemical dosing apparatus is removed, thereby reducing space occupation of the equipment, simplifying the synthesis methanol system, reducing the investment costs, and shortening the construction cycle.

5. The green methanol synthesis apparatus of the present disclosure is compared with the existing synthesis apparatus as follows. As the existing synthesis apparatus performs heat transfer by constant pressure cooling water, when strong reaction heat is rapidly generated in the synthesis apparatus, the existing synthesis apparatus cannot rapidly transfer the heat generated inside because the heat absorbed by the cooling water at a constant pressure is constant. If the pressure of the steam drum is changed, the temperature of the saturated water will rise accordingly, making it impossible to control the internal temperature of the synthesis apparatus. Besides, as the pressure-bearing capacity of the tubes is limited, the existing synthesis apparatus has a weak ability to cope with the temperature rise. In the present disclosure, since the molten salt with a high coefficient of heat conductivity, a high specific heat capacity and a high vaporization point is used, and by an atmospheric-pressure heat transfer and storage method using sensible heat of the molten salt, where an upper limit temperature of the molten salt exceeds 565° C., the heat rapidly generated inside the synthesis apparatus can be stored in the form of sensible heat in the molten salt medium. Then, the heat is rapidly transferred into the outer cavity by circulation, avoiding loss of the internal temperature of the synthesis apparatus and ensuring the stable operation of the internal reaction of the synthesis apparatus.

6. The green methanol synthesis apparatus of the present disclosure is compared with the existing synthesis apparatus as follows. When the carbon monoxide content in the feed gas is high, in order to prevent the internal reaction of the synthesis apparatus from being difficult to control due to the over temperature, part of the carbon monoxide needs to be treated by water vapor conversion in the existing methanol synthesis technology process, in which the carbon monoxide is converted into carbon dioxide and hydrogen, the carbon dioxide is then removed, and then methanol is synthesized from the treated feed gas. In the present disclosure, by using the molten salt with a high specific heat capacity and a wide temperature range as the heat storage medium, the problem that the feed gas cannot be directly introduced into the methanol synthesis apparatus for reaction due to the high content of carbon monoxide can be effectively solved. The generated reaction heat can be stored in the molten salt medium. The molten salt inside the heat storage medium tube bundle of the present disclosure is in an atmospheric-pressure state and has the properties such as low melting point, high boiling point, and large temperature range, and is sufficient to absorb the reaction heat generated rapidly inside the synthesis apparatus, unlike the synthesis apparatus in the conventional art, which cannot bear excessive heat due to the facts that the cooling water is restricted by the pressure of the steam drum and the pressure-bearing capacity of the internal tubes is limited. In the present disclosure, the carbon monoxide conversion process in the existing methanol synthesis process can be omitted, thereby reducing the equipment investment of the conversion process, shortening the investment payoff period, avoiding carbon dioxide emissions in the existing methanol synthesis process, increasing the utilization rate of the feed gas, increasing the methanol yield, and enabling the apparatus having a high conversion rate per pass.

7. The molten salt in the heat storage medium tube bundle of the green methanol synthesis apparatus of the present disclosure flows from top to bottom, and the feed gas enters the inner cavity of the synthesis apparatus through the feed gas distribution assembly and then flows from bottom to top. In such a way, heat exchange is more efficient and heat absorption is more complete. The reaction path of the feed gas flowing along the intermediate axis is long, and the feed gas has sufficient contact with the catalyst, so that the feed gas has sufficient reaction, and the conversion rate per pass of the feed gas is high.

### BRIEF DESCRIPTION OF THE DRAWINGS

FIG. 1 is a schematic structural diagram of an integrated green methanol synthesis apparatus with heat transfer, heat storage and heat exchange by molten salt according to the present disclosure;

FIG. 2 is an entire schematic diagram of an integrated green methanol synthesis apparatus with heat transfer, heat storage and heat exchange by molten salt according to the present disclosure;

FIG. 3 is a schematic structural diagram of a support member, a fixing member according to the present disclosure; and

FIG. 4 is a schematic structural diagram of diagonal braces according to the present disclosure.

In the drawings: 1 reaction shell; 2 heat exchange assembly; 3 cavity; 4 feed gas distribution assembly; 5 floating head; 6 inner barrel; 7 outer barrel; 8 diagonal brace; 9 inner cavity; 10 outer cavity; 11 heat storage medium tube bundle; 12 feed gas tube bundle; 13 lower head; 14 catalyst feed inlet; 15 catalyst discharge outlet; 16 heat storage medium outlet; 17 heat storage medium inlet; 18 feed gas inlet; 19 circulating pump; 20 support member; 21 clamp; 22 intermediate axis; 23 reaction barrel; 24 feed gas reaction outlet; 801 upper pad; 802 diagonal stay; 803 lower pad; 401 feed gas bend tube; 402 feed gas straight tube; 403 feed gas distribution head.

### DETAILED DESCRIPTION OF THE EMBODIMENTS

The present disclosure will be further described below with reference to the specific drawings, but the following embodiments are only preferred embodiments of the present disclosure, not all of them. Based on the embodiments in DETAILED DESCRIPTION OF THE EMBODIMENTS, all the other embodiments that would have been obtained by those skilled in the art without any inventive effort shall fall within the scope of protection of the present disclosure.

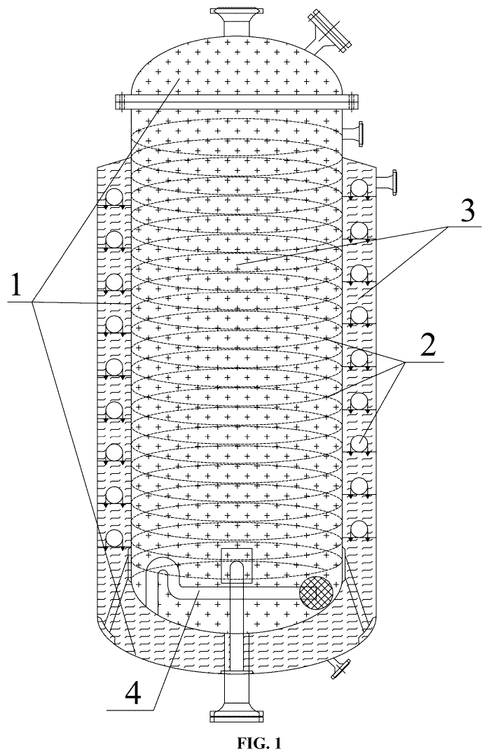

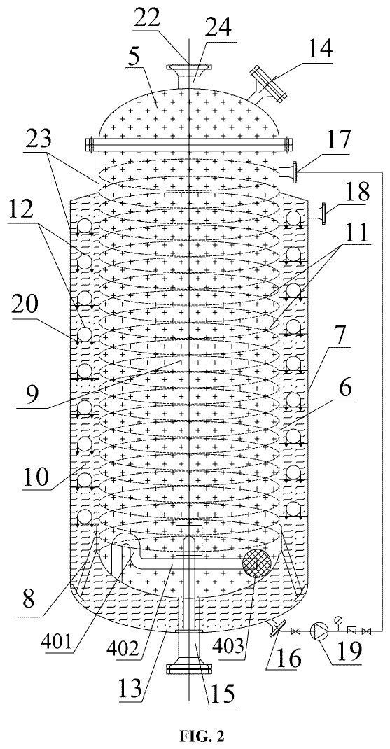

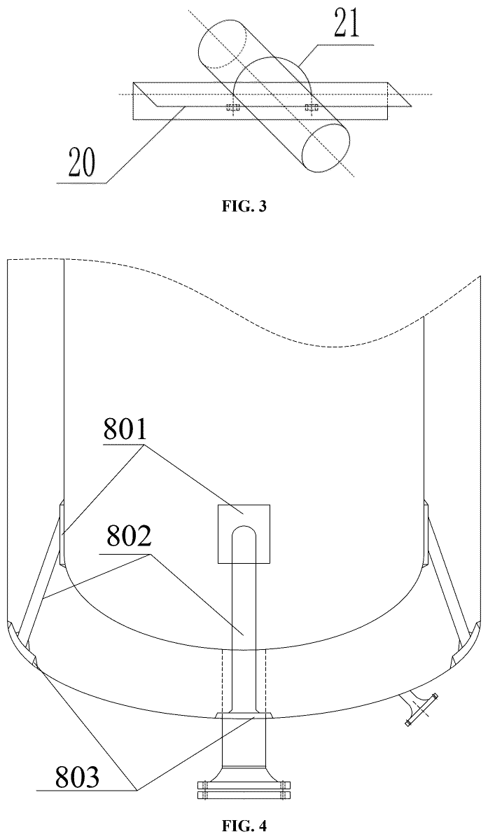

The present disclosure provides an integrated green methanol synthesis apparatus with heat transfer, heat storage and heat exchange by molten salt (see FIGS. 1 to 4), which includes a reaction shell 1, and a heat exchange assembly 2, a cavity 3 and a feed gas distribution assembly 4 disposed inside the reaction shell. The reaction shell 1 includes, from top to bottom, a floating head 5, a reaction barrel 23 and a lower head 13. The floating head 5 is provided with a feed gas reaction outlet 24 and a catalyst feed inlet 14. A catalyst discharge outlet 15 and a heat storage medium outlet 16 are arranged on the lower head 13. The reaction barrel 23 is composed of an inner barrel 6 and an outer barrel 7 as a whole. A feed gas inlet 18 is arranged at the upper end of the outer barrel 7.

The cavity 3 includes an inner cavity 9 formed by an inner wall of the inner barrel 6 and an inner wall of the floating head 5, and an outer cavity 10 formed by an outer wall of the inner barrel 6, an inner wall of the outer barrel 7, and an inner wall of the lower head 13.

Diagonal braces 8 are disposed within the outer cavity to provide a supporting force for the inner barrel, four diagonal braces are uniformly arranged around an outer surface of the inner barrel, and the specific number of the diagonal braces may be set by checking the total weight of the inner barrel and a load inside the inner barrel.

The heat exchange assembly 2 includes a heat storage medium tube bundle 11 in the inner cavity 9 and a feed gas tube bundle 12 in the outer cavity 10.

The feed gas distribution assembly 4 includes a feed gas bend tube 401, a feed gas straight tube 402, and a feed gas distribution head 403 connected by welding, and is located at the bottom of the inner cavity 9 enclosed by the inner barrel 6.

A flange is welded to a lower end of the floating head 5, a counter flange matching with the flange at the lower end of the floating head 5 is welded to an upper end of the inner barrel 6, and the flange of the floating head 5 and the counter flange of the inner barrel 6 fixedly connect the floating head 5 and the inner barrel 6 by means of bolts, nuts and washers, which belongs to a detachable connection. The outer barrel 7 is fixedly welded to and cover an outer layer of the inner barrel 6, and a distance from an upper edge of the outer barrel 7 to an end face of the flange of the inner barrel 6 is 40 cm. A lower end of the outer barrel 6 is rigidly connected to the lower head 13 by welding. The inner barrel 6 is in the form of a cylinder with the lower end closed and the upper end open.

A direction pointing outward from the intermediate axis 22 is defined as from the inside out, and a direction pointing to the intermediate axis 22 is defined as from the outside in.

The feed gas tube bundle 12 is arranged in a coiling form inside the outer cavity 10 from top to bottom, with an upper end being a coil start end and a lower end being a coil tail end. Each circle of the coil is provided with support members 20. Based on an outer surface of the inner barrel 6 as the base, four support members 20 are uniformly arranged around the intermediate axis 22 of the reaction shell 1 at the same height. An included angle between two adjacent support members 20 is 90°. A length of each support member 20 in a radial direction pointing outward from the intermediate axis 22 (close to the side of the outer barrel 7), may be equal to a width of the outer cavity 10 or may extend to the outer side of the tube of the feed gas tube bundle 12. The support members 20 only need to fulfill the function of supporting the feed gas tube bundle 12. Semi-circular clamps 21 are disposed outside the tube of the feed gas tube bundle 12. Two ends of the clamp 21 are provided with threads. Each of the two ends provided with threads passes through the support member 20 and is fixedly connected to the support member 20 via a washer and a nut. The feed gas tube bundle 12 exits from the upper right end of the outer barrel 7 and is connected to the feed gas inlet 18. The coil tail end of the feed gas tube bundle 12 enters the inner cavity 9 of the inner barrel 6 through the bottom of the inner barrel 6, to be connected to the feed gas distribution assembly 4 by welding.

The heat storage medium tube bundle 11 is located in the inner cavity 9 of the inner barrel 6 and arranged in a coiling form from top to bottom along the intermediate axis 22 of the reaction shell 1, with an upper end being a coil start end and a lower end being a coil tail end. The heat storage medium tube bundle 11 is fixed by a method similar to that for the feed gas tube bundle 12. Each circle of the heat storage medium tubes is provided with fixing members (not shown). Based on an inner surface of the inner barrel 6 as the base, four fixing members (not shown) are uniformly arranged around the intermediate axis 22 of the reaction shell 1 at the same height. An included angle between two adjacent fixing members is 90°. A length of each fixing member, in a radial direction from a wall surface of the inner barrel 6 to the intermediate axis 22 (away from the side of the wall surface of the inner barrel 6), may extend to the inner side of the tube of the heat storage medium tube bundle 11. The fixing members only need to fulfill the function of supporting the heat storage medium tube bundle 11. Semi-circular clamps 21 are disposed outside the tube of the heat storage medium tube bundle 11. Two ends of the clamp 21 are provided with threads. Each of the two ends passes provided with threads through the fixing member and is fixedly connected to the fixing member via a washer and a nut. The coil start end of the heat storage medium tube bundle 11 is close to a flange face at the upper end of the inner barrel 6, penetrates the wall surface of the inner barrel 6, to be connected to the heat storage medium inlet 17. The coil tail end of the heat storage medium tube bundle 11 is connected into the outer cavity 10 from the wall surface of the inner barrel 6 on the side close to the bottom of the inner barrel 6.

The heat storage medium outlet 16 is connected to an inlet of a circulating pump 19 via an external pipeline, and an outlet of the circulating pump 19 is connected to the heat storage medium inlet 17 via the external pipeline.

Specifically, both the outer cavity 10 and the interior of the heat storage medium tube bundle 11 are filled with the heat storage medium.

In this embodiment, the outer cavity 10 provides a carrying space for the heat storage medium, and the heat storage medium inside the outer cavity 10 has two main functions: one is to absorb heat transferred by the wall surface of the inner barrel 6; and the other is to transfer the heat absorbed from the inner cavity 9 to the feed gas and exchange heat with the feed gas. The heat storage medium in the heat storage medium tube bundle 11 has two main functions: one is to absorb and transfer the reaction heat of the inner cavity 9 to the outer cavity 10, thus ensuring the reaction temperature of the inner cavity 9; and the other is to store strong reaction heat generated rapidly in the inner cavity 9.

In this embodiment, the feed gas distribution assembly 4 is provided with the feed gas bend tube 401, which can effectively prevent the feed gas from directly reaching the inner cavity 9 to cause the catalyst to clog the feed gas tubes. The feed gas distribution head 403 is installed at a tail end of the feed gas straight tube 402, and has a structural form of a spherical mesh structure which makes the force on its surface uniform and less prone to damage.

In this embodiment, both the feed gas tube bundle 12 and the heat storage medium tube bundle 11 are made of seamless stainless steel tubes. The materials of the seamless stainless steel tubes include but are not limited to 316 stainless steel, 347 stainless steel, and 348 stainless steel. The inner barrel 6, the outer barrel 7, the lower head 13 and the floating head 5 may be made of a material of Q345R or Q370R, but are not limited to the above materials. The material of the reaction shell needs to have properties such as high strength, high heat resistance, oxidation resistance and good weldability.

In this embodiment, a multi-point thermocouple (not shown in the figures) is arranged from top to bottom along the inner surface of the inner barrel 6 and configured to measure an internal temperature of the inner barrel in real time.

Specifically, the circulating pump 19 is a high-temperature-resistant variable-frequency molten salt pump, and the frequency of the circulating pump 19 is controlled by receiving a temperature signal from the multi-point thermocouple, thereby controlling a flow rate of the molten salt in the heat storage medium tube bundle 11 and adjusting a heat transfer rate.

In this embodiment, the multi-point thermocouple of the inner cavity 9 monitors temperature changes in real time when the feed gas is in contact reaction with the catalyst in the inner cavity 9. The multi-point thermocouple transmits the temperature signal to the circulating pump 19 when a sharp change of the temperature inside the synthesis apparatus is detected. A rotational speed of the circulating pump is increased after the circulating pump 19 receives the temperature signal, thereby increasing a rate of circulation of the heat storage medium, further accelerating the transfer of the heat inside the synthesis apparatus, and providing a guarantee for safe operation of the synthesis apparatus.

Further, the diagonal brace 8 includes a lower pad 803, an upper pad 801, and a diagonal stay 802. The lower pad is made as a pad having a curvature consistent with that of an inner surface of the lower head, the lower pad is connected to the lower head by full-length welding. The upper pad is made as a pad having a curvature consistent with that of the outer surface of the inner barrel, the upper pad is connected to the inner barrel by full-length welding. Two ends of the diagonal stay are respectively fittingly welded to the upper pad and the lower pad, and an included angle between the diagonal brace and the outer surface of the inner barrel is 30°-45°.

Further, the heat storage medium inlet is disposed on a side wall of the outer surface of the inner barrel, between the upper edge of the outer barrel and the end face of the flange of the inner barrel, and at an upper right end of the reaction shell. The heat storage medium inlet is welded to a wall surface of the inner barrel.

Further, the feed gas inlet is disposed on a side wall of an outer surface of the outer barrel, at an upper right end of the outer barrel, and directly below the heat storage medium inlet. The feed gas inlet is welded to a wall surface of the outer barrel.

Further, the heat storage medium outlet is located at the lower right side of an outer surface of the lower head, penetrates the lower head and is welded at a position of contact with the lower head. The heat storage medium outlet is located directly below the feed gas inlet and the heat storage medium inlet.

Further, the catalyst feed inlet is located at an angle of 45° on the right side of the floating head. The catalyst feed inlet and the floating head are connected by welding at contact positions. The catalyst feed inlet is blocked by a blind plate during operation of the synthesis apparatus.

Further, the catalyst discharge outlet and the feed gas reaction outlet are installed at the intermediate axis of the reaction shell.

Further, the coil start end of the heat storage medium tube bundle is adjacent to a flange face at the upper end of the inner barrel, penetrates the wall surface of the inner barrel, to be connected to the heat storage medium inlet. The coil tail end of the heat storage medium tube bundle is connected into the outer cavity from the wall surface of the inner barrel on the side close to the bottom of the inner barrel.

The operating principle of the integrated green methanol synthesis apparatus with heat transfer, heat storage and heat exchange by molten salt is as follows.

The feed gas is introduced into the feed gas tube bundle 12 from the feed gas inlet 18, the feed gas spirally flows in the feed gas tube bundle 12 from top to bottom, the high-temperature molten salt inside the outer cavity 10 transfers heat to an outer wall of the feed gas tube bundle 12 by heat convection, the outer wall of the feed gas tube bundle 12 transfers the heat to an inner wall of the feed gas tube bundle 12 by heat conduction to raise the temperature of the inner wall, and the feed gas carries away the heat of the inner wall of the feed gas tube bundle 12 by heat convection, thereby increasing internal energy of the feed gas and raising the temperature of the feed gas. The heated feed gas is introduced into the inner cavity 9 of the reaction shell from the feed gas distribution assembly 4, a methanol gas is produced from the feed gas under the action of the catalyst in the inner cavity 9, the produced methanol gas is discharged from the feed gas reaction outlet 24 at the upper part of the floating head 5 and subjected to a subsequent purification treatment, the release of a large amount of reaction heat accompanies the production of the methanol gas from the feed gas, and the generated reaction heat is stored and absorbed by the molten salt in the heat storage medium tube bundle 11 in the form of sensible heat.

The molten salt stored inside the outer barrel 7 is pumped by means of the circulating pump 19 after heat exchange and is introduced into the heat storage medium tube bundle 11 of the inner cavity 9 through the heat storage medium inlet 17. The reaction heat generated by the reaction of the feed gas in the inner cavity 9 of the reaction shell 1 is transferred to an outer surface of the heat storage medium tube bundle 11 by heat convection, the heat of the outer surface of the heat storage medium tube bundle 11 is transferred to an inner surface by heat conduction, the molten salt spirally flows in the heat storage medium tube bundle 11 from top to bottom to carry away the heat from the inner surface heat storage medium tube bundle 11 by heat convection to enable an increase of internal energy of the molten salt and a rise of the temperature of the molten salt, and the high-temperature molten salt is then introduced from the coil tail end of the heat storage medium tube bundle into the outer cavity 10 to heat the feed gas.

The multi-point thermocouple in the inner cavity 9 monitors temperature changes in real time when the feed gas is in contact reaction with the catalyst in the inner cavity 9, the multi-point thermocouple transmits a temperature signal to the circulating pump 19 when a sharp change of a temperature inside the synthesis apparatus is detected, and a rotational speed of the circulating pump is increased after the circulating pump 19 receives the temperature signal, thereby increasing a rate of circulation of the heat storage medium, further accelerating the transfer of the heat inside the synthesis apparatus, and providing a guarantee for safe operation of the synthesis apparatus.

What is not covered by the present disclosure is applicable to the conventional art.

1-10. (canceled)

11. An integrated green methanol synthesis apparatus with heat transfer, heat storage and heat exchange by molten salt, comprising a reaction shell, and a heat exchange assembly, a cavity and a feed gas distribution assembly disposed inside the reaction shell; wherein the reaction shell comprises, from top to bottom, a floating head, a reaction barrel and a lower head; a feed gas reaction outlet and a catalyst feed inlet are arranged on the floating head; a catalyst discharge outlet and a heat storage medium outlet are arranged on the lower head; the reaction barrel is composed of an inner barrel and an outer barrel as a whole;
the cavity comprises an inner cavity formed by an inner wall of the inner barrel and an inner wall of the floating head, and an outer cavity formed by an outer wall of the inner barrel, an inner wall of the outer barrel and an inner wall of the lower head;
the heat exchange assembly comprises a heat storage medium tube bundle in the inner cavity and a feed gas tube bundle in the outer cavity;
the feed gas distribution assembly is located at a bottom of the inner cavity enclosed by the inner barrel;
a heat storage medium inlet is arranged on the inner barrel, and a heat storage medium is located below a joint between the inner barrel and the floating head and above the outer barrel;
a feed gas inlet is arranged at an upper part of the outer barrel;
the feed gas tube bundle is arranged in a coiling form inside the outer cavity from top to bottom, with an upper end being a coil start end connected to the feed gas inlet, and a lower end being a coil tail end connected to the feed gas distribution assembly within the inner barrel; each circle of a coil of the feed gas tube bundle is fixed to the inner wall of the outer barrel and/or the outer wall of the inner barrel via some of support members;
the heat storage medium tube bundle is located in the inner cavity of the inner barrel and arranged in a coiling form from top to bottom along an intermediate axis of the reaction shell, with an upper end being a coil start end connected to the heat storage medium inlet, and a lower end being a coil tail end passing through the inner barrel to be in communication with the outer cavity; each circle of a coil of the heat storage medium tube bundle is fixed to an inner wall of the inner barrel via some of the support members;
the heat storage medium outlet is connected to an inlet of a circulating pump via an external pipeline, and an outlet of the circulating pump is connected to the heat storage medium inlet via the external pipeline; and both the outer cavity and an interior of the heat storage medium tube bundle are filled with a molten salt heat storage medium;
wherein a flange is welded to a lower end of the floating head, a counter flange matching with the flange at the lower end of the floating head is welded to an upper end of the inner barrel, and the flange of the floating head and the counter flange of the inner barrel fixedly connect the floating head and the inner barrel by means of bolts, nuts and washers, which belongs to a detachable connection; the outer barrel is fixedly welded to and cover an outer layer of the inner barrel; a lower end of the outer barrel is rigidly connected to the lower head by welding; and the inner barrel is in a form of a cylinder with a lower end closed and the upper end open;
wherein the feed gas distribution assembly comprises a feed gas bend tube, a feed gas straight tube, and a feed gas distribution head connected by welding, the feed gas bend tube is connected to the coil tail end at the lower end of the feed gas tube bundle;
wherein a straight tube section of the catalyst discharge outlet passes through the lower head and a bottom end of the inner barrel in sequence and is in communication with the inner cavity; a catalyst is filled in the inner cavity formed by the inner wall of the inner barrel and the inner wall of the floating head, with a packing factor up to 80%.
wherein a bottom of the inner barrel is constructed as a dome structure to facilitate discharging of a catalyst;
the heat storage medium tube bundle, the outer cavity, the circulating pump and the external pipeline together form a heat storage medium circulation system, the heat storage medium is configured for heating a feed gas in the outer cavity by absorbing reaction heat in the inner cavity, an entire heating process occurs in an independent synthesis apparatus without a provision of an external preheater and an external heater;
wherein an operating principle of the green methanol synthesis apparatus is as follows:
the feed gas is introduced into the feed gas tube bundle from the feed gas inlet, the feed gas spirally flows in the feed gas tube bundle from top to bottom, the molten salt with high temperature inside the outer cavity transfers heat to an outer wall of the feed gas tube bundle by heat convection, the outer wall of the feed gas tube bundle transfers the heat to an inner wall of the feed gas tube bundle by heat conduction to raise a temperature of the inner wall, and the feed gas carries away the heat of the inner wall of the feed gas tube bundle by heat convection to increase internal energy of the feed gas to raise a temperature of the feed gas; the feed gas after heated is introduced into the inner cavity of the reaction shell from the feed gas distribution assembly, a methanol gas is produced from the feed gas under an action of a catalyst in the inner cavity, the produced methanol gas is discharged from the feed gas reaction outlet at an upper part of the floating head and subjected to a subsequent purification treatment, release of a large amount of the reaction heat accompanies a production of the methanol gas from the feed gas, and the reaction heat is stored and absorbed by the molten salt heat storage medium in the heat storage medium tube bundle in a form of sensible heat;
the molten salt heat storage medium stored inside the outer barrel is pumped by means of the circulating pump after heat exchange and is introduced into the heat storage medium tube bundle of the inner cavity through the heat storage medium inlet; the reaction heat generated by a reaction of the feed gas in the inner cavity of the reaction shell is transferred to an outer surface of the heat storage medium tube bundle by heat convection, the heat of the outer surface of the heat storage medium tube bundle is transferred to an inner surface of the heat storage medium tube bundle by heat conduction, the molten salt heat storage medium spirally flows in the heat storage medium tube bundle from top to bottom to carry away the heat from the inner surface of the heat storage medium tube bundle by heat convection to enable an increase of internal energy of the molten salt heat storage medium and a rise of a temperature of the molten salt heat storage medium, and the molten salt heat storage medium with high temperature is then introduced from the coil tail end of the heat storage medium tube bundle into the outer cavity to heat the feed gas;
by using the coiling form, the heat storage medium in a tube bundle is in a normal pressure state, and tubes in the tube bundle are subjected to a small pressure and capable of effectively relieving thermal stress through rotation curvature;
a multi-point thermocouple in the inner cavity monitors temperature changes in real time when the feed gas is in contact reaction with the catalyst in the inner cavity, the multi-point thermocouple transmits a temperature signal to the circulating pump when a sharp change of a temperature inside the synthesis apparatus is detected, and a rotational speed of the circulating pump is increased after the circulating pump receives the temperature signal, thereby increasing a rate of circulation of the molten salt heat storage medium, further accelerating a transfer of the heat inside the synthesis apparatus, and providing a guarantee for safe operation of the synthesis apparatus.

12. The integrated green methanol synthesis apparatus with heat transfer, heat storage and heat exchange by molten salt according to claim 11, wherein a distance from an upper edge of the outer barrel to an end face of the flange of the inner barrel is 30 cm-50 cm.

13. The integrated green methanol synthesis apparatus with heat transfer, heat storage and heat exchange by molten salt according to claim 11, wherein the feed gas distribution head is of a spherical mesh structure.

14. The integrated green methanol synthesis apparatus with heat transfer, heat storage and heat exchange by molten salt according to claim 11, wherein ones of the support members at a same height are uniformly distributed in a circumferential direction, the support members have horizontal support surfaces, and the coils are fixed onto the horizontal support surfaces by means of semicircular clamps; and each of the support members within the outer cavity has a length equal to a width of the outer cavity or extending from the outer wall of the inner barrel to an outer side of a corresponding one of tubes of the coil of the feed gas tube bundle, each of the support members within the inner cavity has a length extending from the inner wall of the inner barrel to an inner side of a corresponding one of the tubes of the coil of the heat storage medium tube bundle, and the support members are capable of supporting the feed gas tube bundle/the heat storage medium tube bundle.

15. The integrated green methanol synthesis apparatus with heat transfer, heat storage and heat exchange by molten salt according to claim 11, wherein the straight tube section of the catalyst discharge outlet is connected with the lower head and the bottom end of the inner barrel by welding at contact positions; the catalyst discharge outlet is blocked by means of a blind plate during operation of the synthesis apparatus.

16. The integrated green methanol synthesis apparatus with heat transfer, heat storage and heat exchange by molten salt according to claim 11, wherein diagonal braces are disposed within the outer cavity to provide a supporting force for the inner barrel, the diagonal braces are uniformly arranged around an outer surface of the inner barrel, and a specific number of the diagonal braces is set by checking a total weight of the inner barrel and a load inside the inner barrel; and
each of the diagonal braces comprises a lower pad, an upper pad, and a diagonal stay, the lower pad is made as a pad having a curvature consistent with that of an inner surface of the lower head, the lower pad is connected to the lower head by full-length welding, the upper pad is made as a pad having a curvature consistent with that of the outer surface of the inner barrel, the upper pad is connected to the inner barrel by full-length welding, and two ends of the diagonal stay are fittingly welded to the upper pad and the lower pad respectively.

17. The integrated green methanol synthesis apparatus with heat transfer, heat storage and heat exchange by molten salt according to claim 16, wherein an included angle between the diagonal brace and the outer surface of the inner barrel is 30°-45°.

18. The integrated green methanol synthesis apparatus with heat transfer, heat storage and heat exchange by molten salt according to claim 11, wherein the heat storage medium is the molten salt of a binary salt or a ternary salt.

19. The integrated green methanol synthesis apparatus with heat transfer, heat storage and heat exchange by molten salt according to claim 11, wherein an interval between upper and lower adjacent tubes in a same type of tube bundles of the feed gas tube bundle and the heat storage medium tube bundle is of 3 cm-6 cm.

20. The integrated green methanol synthesis apparatus with heat transfer, heat storage and heat exchange by molten salt according to claim 11, wherein a multi-point thermocouple is arranged from top to bottom along an inner surface of the inner barrel and configured to measure an internal temperature of the inner barrel in real time; and the circulating pump is a high-temperature-resistant variable-frequency molten salt pump, and a frequency of the circulating pump is controlled by receiving temperature signals from the multi-point thermocouple to control a flow rate of the molten salt in the heat storage medium tube bundle and adjust a heat transfer rate.
